(19) United States
(12) Patent Application Publication
(10) Pub. No.: US 2005/0075577 A1
(43) Pub. Date: Apr. 7, 2005

(54) Wireless heart rate monitor with infrared detecting module

(76) Inventor: Yu-Yu Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/679,304

(22) Filed: Oct. 7, 2003

(51) Int. Cl. A61B 5/0402

(52) U.S. Cl. 600/509

(57) ABSTRACT

A wireless heart rate monitor includes a main body in which an infrared detecting module and a radio frequency transmitter are fixed and a separate radio frequency receiver is positioned at a remote site from the main body. The infrared detecting module includes an infrared transmitter and an infrared receiver, which cooperate together to detect the pulsing condition of a person when the main body is positioned on a suitable location of the user's skin and generates a heartbeat signal associated with the pulsing condition. The heartbeat signal is transmitted as a radio frequency signal by the radio frequency transmitter and the radio frequency signal is received by the radio frequency receiver and processed by a microprocessor based device to generate a display signal that is then displayed on a display for visual observation by the user or other persons. The location of the radio frequency receiver is not subject to any limitation as long as it is located within a transmission range of the radio frequency transmitter whereby the radio frequency receiver can be worn on the wrist of the user or mounted on fitness equipment by which the user is taking exercise.

### BACKGROUND OF THE INVENTION

1. Field of the Invention

The present invention relates generally to a heartbeat sensing device, and in particular to a wireless heart rate monitor with infrared detecting module.

2. Description of the Prior Art

Heartbeat sensing devices or heart rate monitors are used to detecting the heartbeat of a human body, especially a person who is doing exercise, or for medical use. For example, the heartbeat sensing device can be integrated with fitness equipment whereby when a person does exercise with such fitness equipment, the pulsing condition of the person can be timely detected for monitoring the body condition of the exercising person. Often a display is employed with the heartbeat sensing device for display of the pulsing condition of the person.

The heartbeat sensing device is comprised of conductive rubber pads attached to a human body to generate a heartbeat signal and a control circuit capable of receiving, processing and displaying the heartbeat signal. Electrical connection is provided between the conductive rubber pads and the control circuit whereby the heartbeat of the person is detected by the rubber pads and transmitted as a heartbeat signal to the control circuit where the heartbeat signal is processed in accordance with pre-established processing algorithm. The result of the processing is then displayed on the display for visual observation.

Wireless heartbeat sensing devices are also available. The wireless heartbeat sensing device comprises a wireless transmitter circuit, which transmits the heartbeat signal to a receiver in a remote site within a predetermined effective range.

Conventionally, the heartbeat sensing device is made as a chest mounting device that is attached to the skin of the chest for detecting the pulsing condition. This is generally unhandy to most users.

A wristwatch type heartbeat sensing device is also known, which is made as a wristwatch that can be worn on a wrist of a person. Electrode means of the wristwatch type heartbeat sensing device is arranged on a top or side surface of the heartbeat sensing device whereby the other hand of the user is required to contact the electrode means in order to perform heartbeatdetection. This is troublesome for people taking outdoor activities, such as bicycle riding, jogging and skiing.

Another wristwatch type heartbeat sensing device makes use of infrared light to detect the pulsing condition of a wearer. However, to properly detect the pulsing condition, an infrared heartbeat sensing unit is mounted at the underside of a watchband and has to be positioned at a location of the wrist where the pulsing signal is the strongest. Since the wrist size is different from person to person, it is difficult to have the heartbeat sensing unit precisely located on the point where the pulsing is the strongest for each particular person. In addition, signal transmission between the heartbeat sensing unit and the wristwatch of the heartbeat sensing device is provided by conductors extending therebetween. Waterproofness is difficult to realize in outdoor activities.

Thus, it is desired to have a heartbeat sensing device that overcomes the problems and drawbacks encountered in the prior arts.

### SUMMARY OF THE INVENTION

In view of the drawbacks of the conventional heartbeat sensing devices, an object of the present invention is to provide a wireless heart rate monitor comprising a built-in infrared detecting module directly positionable on the point of skin where the pulsing is the strongest to directly detect the pulsing condition and a receiver unit located at a remote site for receiving, in a wireless manner, a heartbeat signal from the infrared detecting module, which heartbeat signal is processed and displayed at the remote site.

Another object of the present invention is to provide a heart rate monitor that has flexibility in practical applications wherein an infrared detecting module is positionable on a wrist or a temple of a wearer, while a receiver unit is positionable in a remote site, such as fitness equipment, for receiving and displaying results of pulsing detection.

To overcome the deficiencies of the prior art, the present invention provides a wireless heart rate monitor with infrared detecting module. The heart rate monitor comprises a main body in which an infrared detecting module and a radio frequency transmitter are fixed and a separate radio frequency receiver is positioned at a remote site from the main body. The infrared detecting module comprises an infrared transmitter and an infrared receiver, which cooperate together to detect the pulsing condition of a person when the main body is positioned on a suitable location of the user's skin and generates a heartbeat signal associated with the pulsing condition. The heartbeat signal is transmitted as a radio frequency signal by the radio frequency transmitter and the radio frequency signal is received by the radio frequency receiver and processed by a microprocessor based device to generate a display signal that is then displayed on a display for visual observation by the user or other persons. The location of the radio frequency receiver is not subject to any limitation as long as it is located within a transmission range of the radio frequency transmitter whereby the radio frequency receiver can be worn on the wrist of the user or mounted on a fitness equipment by which the user is taking exercise.

### BRIEF DESCRIPTION OF THE DRAWINGS

The present invention will be apparent to those skilled in the art by reading the following description of a preferred embodiment thereof, with reference to the attached drawings, in which:

FIG. 1 is a top plan view of a wireless heart rate monitor with infrared detecting module constructed in accordance with a preferred embodiment of the present invention;

FIG. 2 is a sectional view taken along line 2-2 of FIG. 1;

FIG. 3 is a bottom view of the wireless heart rate monitor of the present invention;

FIG. 4 is a schematic view showing the wireless heart rate monitor of the present invention worn on a wrist of a user, while a receiver unit is worn on another hand of the user;

FIG. 5 is a schematic view showing the wireless heart rate monitor of the present invention worn on head of a user, while a receiver unit is worn on a wrist of the user;

FIG. 6 is a schematic view showing the wireless heart rate monitor of the present invention worn on a wrist of a user, while a receiver unit is mounted to an indoor jogging machine;

FIG. 7 is a schematic view showing the wireless heart rate monitor of the present invention worn on a wrist of a user, while a receiver unit is mounted to a stationary bicycle;

FIG. 8 is a schematic view showing the wireless heart rate monitor of the present invention worn on a wrist of a user, while a receiver unit is mounted to a regular bicycle; and

FIG. 9 is a block diagram of a control circuit of the wireless heart rate monitor of the present invention.

### DETAILED DESCRIPTION OF PREFERRED EMBODIMENT

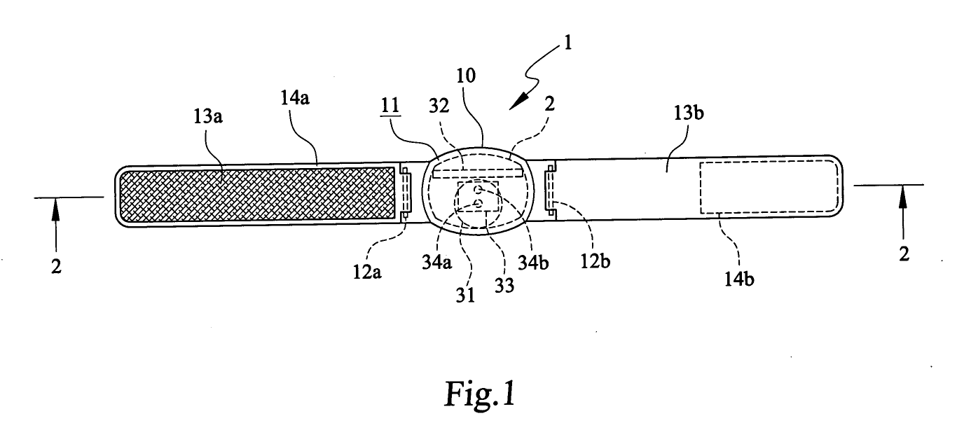

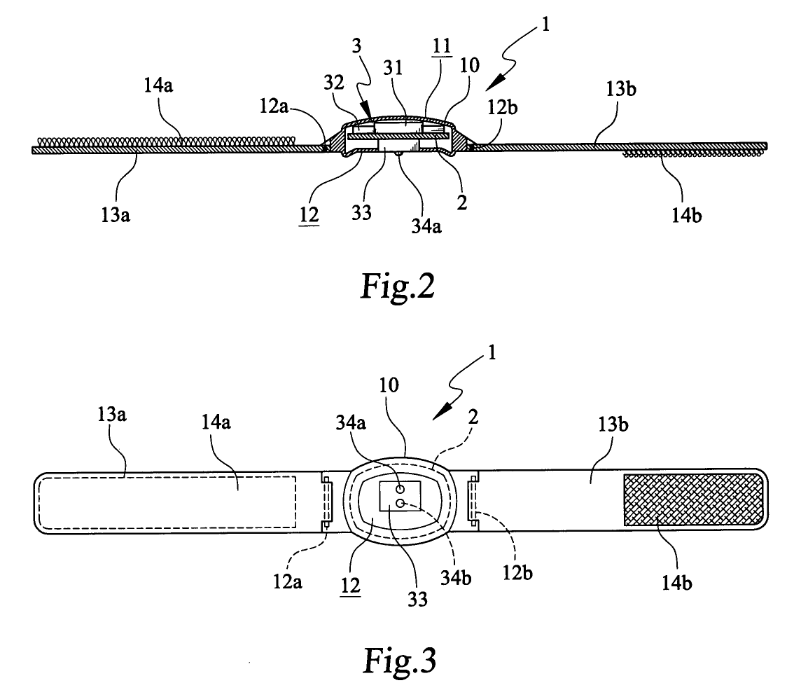

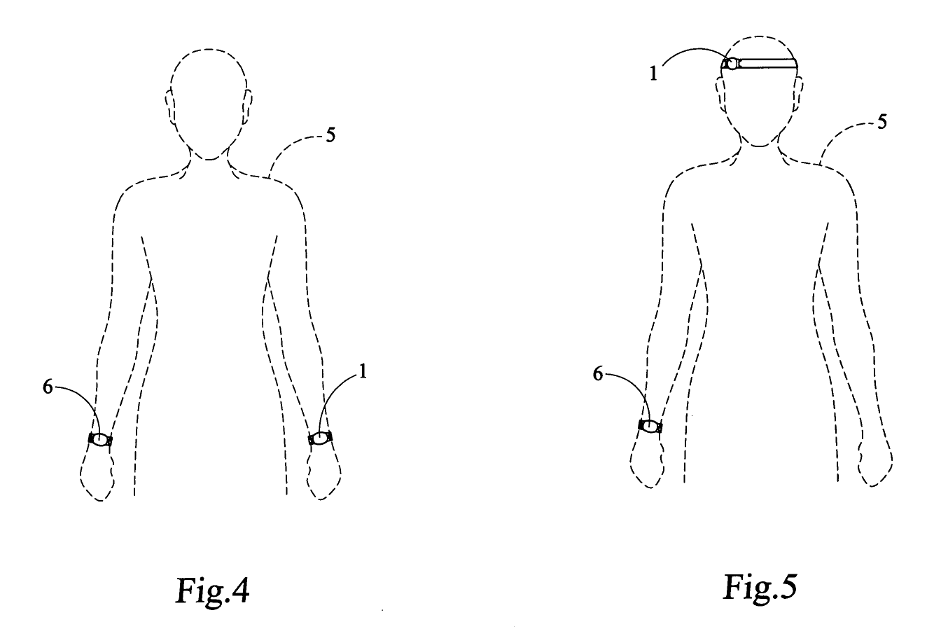

With reference to the drawings and in particular to FIGS. 1-3, a wireless heart rate monitor constructed in accordance with a preferred embodiment of the present invention, generally designated with reference numeral 1, comprises a main body 10 in the form of a wristwatch having a bezel or top cover 11 facing away from the skin of a user 5 and a back cover 12 positionable on and physically engaging the skin of the user 5 (see FIGS. 4-8). Pivot members 12a, 12b are provided on opposite edges of the main body 10 for connection with bands 13a, 13b, which allow the user to wear the main body 10 on for example a wrist. The bands 13a, 13b are made of resilient material or are made extendible whereby the main body 10 can be securely fixed on the wrist. The bands 13a, 13b can be replaced by elastic strips that are of different lengths so that the main body 10 is mountable on any desired portion of a human body, such as the head as shown in FIG. 5.

Fastening elements, such as Velcro strips or pads 14a, 14b, are formed on the bands 13a, 13b or the elastic strips for fastening the main body 10 on the human body. It is apparent that different types of fastening elements can also be employed to fasten the main body 10 on the human body.

A circuit board 2 is fixed inside the main body 10. A control circuit 3 comprising a power source, such as a battery set 31, and a transmission antenna 32, as well as other electronic parts, is formed on the circuit board 2. The control circuit 3 will be further discussed.

An infrared detecting module 33 is formed on a bottom surface of the circuit board 2. The infrared detecting module 33 comprises an infrared transmitter unit 34a and an infrared receiver unit 34b, which cooperate together to sense the pulsing condition of the user 5. The infrared detecting module 33 is exposed, at least optically, through the back cover 12 of the main body 10 whereby when the main body 10 is fastened on the wrist of the user 5, such as that shown in FIG. 4, the infrared detecting module 33 engages the skin of the user 5 with the infrared transmitter 34a and the infrared receiver 34b substantially located on the point where the pulsing is the strongest. The infrared detecting module 33 thus detects and reads the heart rate of the user 5.

The technique of using infrared light to detect the pulsing condition of a person is known and thus no further detail will be given herein.

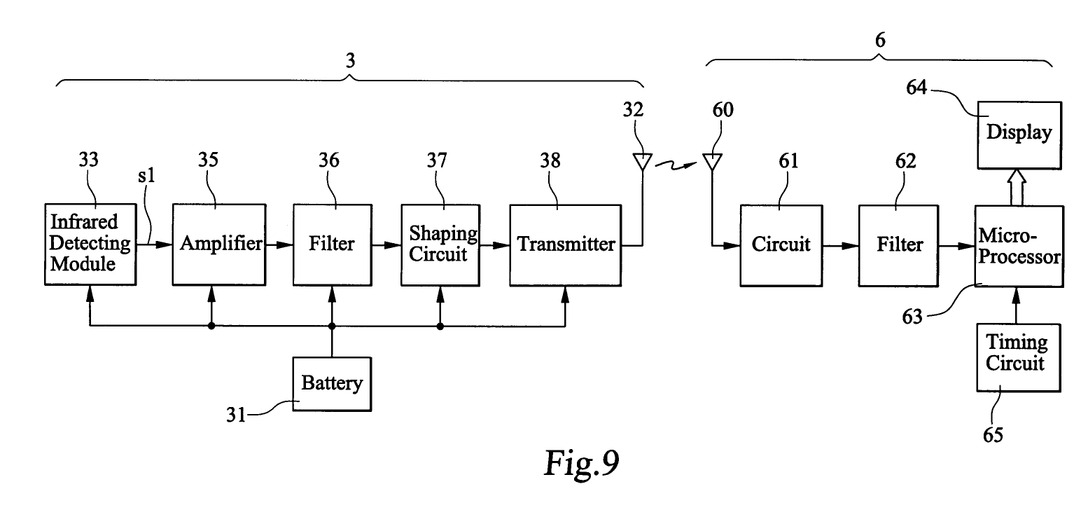

A heartbeat signal provided by the infrared detecting module 33, associated with the pulsing condition of the user 5, is transmitted in a wireless manner by means of for example radio frequency signal, to a receiver unit 6 that is mounted on for example the wrist of another hand of the user 5 as illustrated in FIG. 4. The heartbeat signal that is received by the receiver unit 6 is processed by the receiver unit 6 and a display signal is issued and displayed on a display 64 (see FIG. 9) associated with the receiver unit 6.

FIG. 5 shows another application of the wireless heart rate monitor 1 of the present invention wherein the main body 10 of the wireless heart rate monitor 1 is mounted on the head of the user 5 with the infrared detecting module 33 substantially positioned on the temple of the user 5 for detection of the pulsing occurring in the temple. The heartbeat of the user 5 is thus detected by the infrared transmitter 34a and the infrared receiver 34b. Similarly, the heartbeat signal of the infrared detecting module 33 is transmitted in radio frequency to the receiver unit 6 that is mounted on a wrist of the user 6 for ready reading for the user 5.

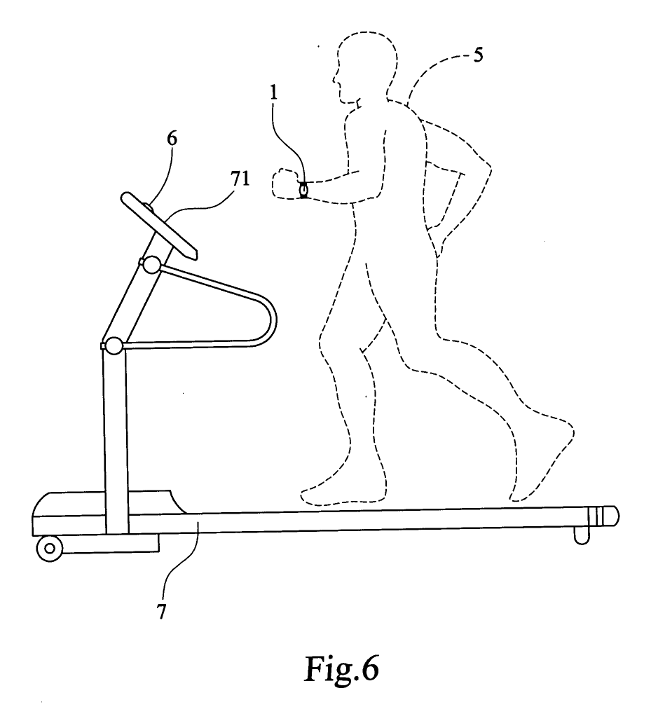

FIG. 6 shows a further application of the wireless heart rate monitor 1 of the present invention wherein the main body 10 in which the wireless heart rate monitor 1 is arranged is worn on a wrist of the user 5, while the receiver unit 6 is mounted on a panel 71 of a fitness equipment 7, such as a jogging machine. Thus, when the user 5 is jogging on the jogging machine 7, the pulsing condition of the user 5 is detected by the infrared detecting module 33 of the main body 10 and transmitted to the receiver unit 6. The reading of the heartbeat is displayed on the receiver unit 6 mounted on the panel 7 for ready observation.

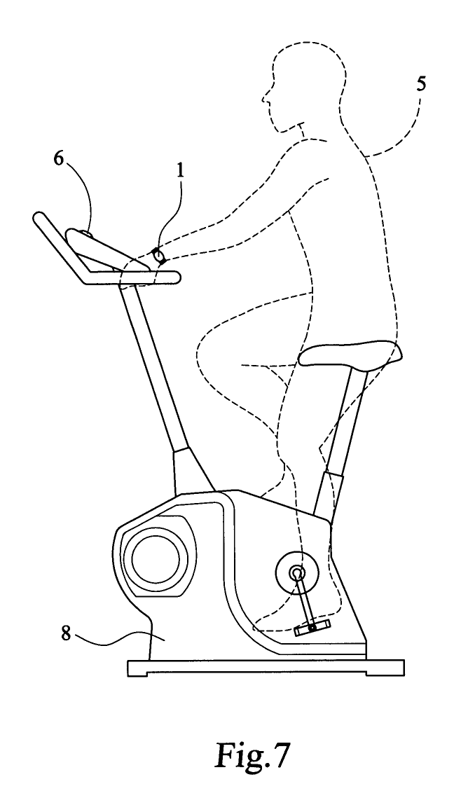

FIG. 7 shows a further application of the wireless heart rate monitor 1 of the present invention wherein the main body 10 in which the wireless heart rate monitor 1 is arranged is worn on a wrist of the user 5, while the receiver unit 6 is mounted on indoor exercising equipment 8, such as a stationary bicycle. Thus, when the user 5 is riding the stationary bicycle 8, the pulsing condition of the user 5 is detected by the infrared detecting module 33 of the main body 10 and transmitted to the receiver unit 6. The reading of the heartbeat is displayed on the receiver unit 6 fixed at a suitable position in front of the user 5 for ready observation.

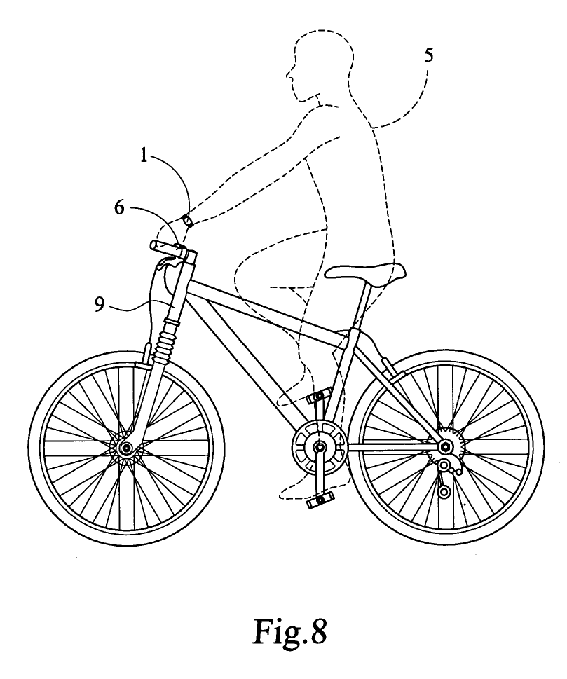

FIG. 8 shows yet a further application of the wireless heart rate monitor 1 of the present invention wherein the main body 10 in which the wireless heart rate monitor 1 is arranged is worn on a wrist of the user 5, while the receiver unit 6 is mounted on a handlebar of a regular bicycle 9. Thus, when the user 5 is riding the bicycle 9, the pulsing condition of the user 5 is detected by the infrared detecting module 33 of the main body 10 and transmitted to the receiver unit 6. The reading of the heartbeat is displayed on the receiver unit 6 for ready observation by the user 5.

Also referring to FIG. 9, which is a block diagram of the control circuit 3 of the wireless heart rate monitor 1 of the present invention, the control circuit 3 comprises the infrared detecting module 33 that detects the pulsing condition of the user 5 by means of the infrared transmitter 34a and the infrared receiver 34b to obtain a heartbeat signal s1. The heartbeat signal s1 is forwarded to, in sequence, an amplifier 35 for amplification, a filter 36 for filtering the noise, and a shaping circuit 37 for shaping, and then applied to a radio frequency transmitter 38. The transmitter 38 transmits the heartbeat signal s1 through the transmission antenna 32 as a radio frequency signal.

The radio frequency signal is transmitted to the receiver unit 6. The receiver unit 6 comprises a receiving antenna 60 and an associated receiver circuit 61 that receives the radio frequency signal. The received radio frequency signal is filtered by a filter 62 and then applied to a microprocessor 63. The microprocessor 63 processes the received signal to issue the display signal that is then displayed on a display 64. The receiver unit 6 further comprises a timing circuit 65, which provides a timing signal to the microprocessor 63 and the display 64. This allows the wireless heart rate monitor 1 to function as a timer as well.

The microprocessor 63 can be provided with suitable algorithm and programmed to perform other functions, such as calculation of calories that are consumed by the user 5 when taking an exercise based on the pulsing condition and other parameters that may be pre-loaded therein or entered by an option input device.

Although the present invention has been described with reference to the preferred embodiment thereof and the best mode for controlling the operation of the fuel cells, it is apparent to those skilled in the art that a variety of modifications and changes may be made without departing from the scope of the present invention which is intended to be defined by the appended claims.

1. A wireless heart rate monitor comprising:
a main body having a top cover and a back cover adapted to be positioned on the skin of a user;
an infrared detecting module mounted in the main body and comprising an infrared transmitter and an infrared receiver exposed through the back cover to engage the skin for detecting pulsing condition of the user and issuing a heartbeat signal;
a radio frequency transmitter for receiving the heartbeat signal and transmitting the heartbeat signal as a radio frequency signal; and
a radio frequency receiver for receiving and processing the radio frequency signal to provide a reading of the pulsing condition of the user.

2. The wireless heart rate monitor as claimed in claim 1, wherein the wireless heart rate monitor is formed as a wristwatch, the main body further comprising bands extending from opposite edges of the main body user for wearing the wireless heart rate monitor on the user's wrist.

3. The wireless heart rate monitor as claimed in claim 2 further comprising fastening means on the bands for fastening the main body on the user.

4. The wireless heart rate monitor as claimed in claim 1 further comprising elastic strips attached to opposite edges of the main body for fastening the main body on head of the user.

5. The wireless heart rate monitor as claimed in claim 4 further comprising fastening means on the strips for fastening the main body on the user.

6. The wireless heart rate monitor as claimed in claim 1, wherein the radio frequency receiver comprises:
a radio frequency receiver circuit for receiving the radio frequency signal transmitted by the radio frequency transmitter;
a microprocessor for receiving and processing the radio frequency received by the receiver circuit and issuing a display signal; and
a display for receiving and displaying the display signal.

7. The wireless heart rate monitor as claimed in claim 6, wherein the radio frequency receiver further comprises a timing circuit which provides a timing signal to the microprocessor and the display whereby the wireless heart rate monitor functions as a timer as well.

8. The wireless heart rate monitor as claimed in claim 7, wherein microprocessor comprises an algorithm for calculation calories consumed by the user based on the heartbeat signal and timing signal.

9. The wireless heart rate monitor as claimed in claim 6, wherein the radio frequency receiver is adapted to be worn on a wrist of the user.

10. The wireless heart rate monitor as claimed in claim 6, wherein the radio frequency receiver is adapted to be mounted on a fitness equipment.

11. The wireless heart rate monitor as claimed in claim 6, wherein the radio frequency receiver is adapted to be mounted on an outdoor exercising device.
